(19) United States
(12) Patent Application Publication
(10) Pub. No.: US 2016/0090617 A1
(43) Pub. Date: Mar. 31, 2016

(54) CONCENTRATION DEVICE FOR MICROORGANISMS IN LARGE VOLUMES OF TURBID WATER AND METHOD THEREFOR

(71) Applicant: U.S. ENVIRONMENTAL PROTECTION AGENCY, Washington, DC (US)

(72) Inventor: VICENTE JAMES GALLARDO, CINCINNATI, OH (US)

(21) Appl. No.: 14/496,887

(22) Filed: Sep. 25, 2014

(51) Int. Cl. C12Q 1/24

(52) U.S. Cl. CPC C12Q 1/24

(57) ABSTRACT

A device and method for concentrating microorganisms in large volumes of turbid water. When particulate builds up at the inlet filter header and pressure increases, filtrate that has collected within the filtrate reservoir serves as backflush fluid. The hollow fiber filter cartridge is backflushed as the pump reverses and filtrate flows from the filtrate reservoir, back through the hollow fiber filter cartridge, and out the inlet filter header, thereby dislodging the accumulated particulate. Elution solution is pumped though the device to remove microorganisms that may have adhered to the filter fibers and tubing. The retentate is then recovered by forward flushing the filter with eluting solution so that the retentate is sent to the off-line retentate sample container. There, the retentate sample is isolated and prevented from re-entering the hollow fiber filter cartridge. The inlet filter header therefore serves as an in-line pre-filter.

### FIELD OF THE INVENTION

The disclosure generally relates to water sampling and analysis, and more particularly, to a device and method for concentrating water samples ranging from low to high turbidity.

### BACKGROUND OF THE INVENTION

Identifying waterborne pathogens accurately and quickly is critical for public notification and remediation decision-making. In some situations, it is necessary to concentrate highly turbid water samples; e.g. wash down water that is generated during building decontamination of Bacillus anthracis spores, surface water samples that are collected for Cryptosporidium parvum (C. parvum) and Giardia, beach water samples collected for human health pathogens, irrigation water for food outbreak prevention and/or investigation, etc.

The Environmental Protection Agency (EPA) has previously developed a Water Sample Concentrator (WSC) to concentrate microorganisms in drinking water samples. The WSC has been used to integrate and automate the processing of water samples and concentration of microbial material without destroying the pathogens. The WSC is a semi-automatic device that allows an operator with minimal training to safely concentrate a 100 Liter sample in the field and send the resulting concentrated sample (typically 1 Liter or less) to a laboratory for analysis. The WSC also has a fluid path that can be removed after a sample has been processed and replaced with a fresh fluid path apparatus. This allows an operator to concentrate numerous samples during a single field trip with no danger of cross-contamination between sample runs. The WSC was developed for detecting low levels of highly pathogenic organisms. Other systems, typically also using the principles of hollow fiber ultrafiltration, have also been developed for concentrating microorganisms in large (40-100 Liter) water samples.

The WSC and those other technologies have been effective for concentrating drinking water samples; i.e. samples containing very low levels of particulate matter. However, if samples contain significant amounts of particulate matter, the particulate matter tends to clog at the inlet header of the filter. Typically, it is not the individual pores along the length of the fibers that clog, but the entrances to the hollow fibers which clog. The filters used in those other technologies are typically single-use filters due to the desire to lessen the probability of cross-contamination between sample concentration runs. Those types of filters are compact, economical, and provide a high filter surface area. But, the diameter of the individual fibers is small; e.g. 0.180 to 0.200 mm. Particulate matter larger than the fiber diameter tend to accumulate at the entrance to these fibers causing operational problems.

Pre-filters may be used to filter out gross particulate matter to keep the filter clean; however, this greatly increases the suction head of the pump. The pumps typically used in those other technologies are peristaltic pumps, which allow a replaceable fluid path; however, those pumps perform poorly with increasing suction head. Furthermore, the material that is trapped in a pre-filter would likely contain a percentage of the target organisms and thus would partially defeat the purpose for concentrating the water sample. Even the use of a pre-filter with the WSC did not prevent the inlet filter header from clogging. The mesh size of the pre-filter was small enough to capture particles larger than the hollow fiber's inside diameter. But, the filter still clogged, likely due to smaller particles passing through the pre-filter and agglomerating to a larger size and causing clogging of the filter. In the WSC, this agglomeration may occur in the retentate sample bottle.

Tangential flow filtration systems, like those used in dialysis and water desalinization, have also been developed to help minimize clogging or fouling of filter pores. However, those systems focus on the reduction of clogging or fouling that occurs at the microscopic pores along the filter surface rather than on reducing clogging caused by particulate matter of much larger size that occurs at the entrances to the filter fibers.

Other methods for preventing clogging have been developed wherein certain concentration systems are cleaned in between runs. But, these systems are often too complex for field deployment. Dead end filtration methods have also been used, but that often leads to lower recovery of microorganisms.

Therefore, it would be desirable to provide a device and method that will overcome the aforementioned problems.

### SUMMARY OF THE INVENTION

This summary is provided to introduce a selection of concepts in a simplified form that are further described below in the DESCRIPTION OF THE APPLICATION. This summary is not intended to identify key features of the claimed subject matter, nor is it intended to be used as an aid in determining the scope of the claimed subject matter.

In accordance with one embodiment of the present invention, a device for concentrating microorganisms in a water sample is disclosed. The device comprises: a line of tubing; a reversible pump; a filter; an inlet filter header coupled to an inlet of the filter; a plurality of valves for controlling the flow of liquids through the tubing; and a filtrate reservoir for collecting filtrate after the filtrate has exited a filtrate port of the filter, wherein the inlet filter header is adapted to be used as an in-line pre-filter for the device.

In accordance with another embodiment of the present invention, a method for concentrating microorganisms in a water sample is disclosed. The method comprises the steps of: providing a device for concentrating microorganism in a water sample; pumping the water sample through the device in a forward direction; collecting particulate matter from the water sample within an in-line pre-filter; backflushing the pre-filter with filtrate pumped through the device in a reverse direction; collecting retentate in an off-line retentate sample container, wherein the retentate contains the particulate matter; and isolating the particulate matter within the off-line retentate sample container in order to prevent the particulate matter from being recirculated through the device.

In accordance with another embodiment of the present invention, a method for concentrating microorganisms in a water sample is disclosed. The method comprises the steps of: providing a device for concentrating microorganisms in a water sample, the device comprising: a line of tubing; a variable speed, reversible peristaltic pump; a hollow fiber filter cartridge; an inlet filter header coupled to an inlet of the filter; a plurality of valves for controlling the flow of liquids through the tubing; a filtrate reservoir; and a retentate sample container coupled off-line to the device for collecting retentate; priming the device with pretreatment solution in order to remove air from the device; recirculating the pretreatment solution through the device in order to coat filter fibers and tubing with a hydrophilic solution that will prevent the microorganisms from sticking to the filter fibers and to the tubing; pumping the water sample through the device in a forward direction; collecting an amount of filtrate within the filtrate reservoir after the water has exited a filtrate port of the hollow fiber filter cartridge; backflushing the filtrate in a reverse direction from the filtrate reservoir back through the hollow fiber filter cartridge and out the inlet filter header in order to dislodge particulate matter that has collected at the inlet filter header; collecting retentate within the retentate sample container; and pumping hydrophilic eluting solution through the device.

### BRIEF DESCRIPTION OF THE DRAWINGS

FIG. 1 a simplified functional block diagram of a system implementing a device for concentrating microorganisms in large volumes of turbid water in accordance with one or more aspects of the present invention;

FIG. 2 is a diagram of the device for concentrating microorganisms in large volumes of turbid water in accordance with one or more aspects of the present invention;

FIG. 3 is the device of FIG. 2 wherein a pretreatment solution is being drawn up into the device and wherein air is purged from the device;

FIG. 4 is the device of FIG. 2 wherein the pretreatment solution is being recirculated;

FIG. 5 is the device of FIG. 2 wherein the pretreatment solution is being removed;

FIG. 6 is the device of FIG. 2 wherein a large volume water sample is being drawn up into the device;

FIG. 7 is the device of FIG. 2 wherein the device is being backflushed;

FIG. 8 is the device of FIG. 2 wherein eluting solution is being drawn up into the device;

FIG. 9 is the device of FIG. 2 wherein the retentate sample is recovered from the device;

FIG. 10 is the device of FIG. 2 wherein a collapsible retentate container is used with the device;

FIG. 11 is the device of FIG. 10 wherein the device is being backflushed using a collapsible retentate container; and

FIG. 12 is a diagram comparing a prior art device with the device of the present invention.

### DETAILED DESCRIPTION OF THE PREFERRED EMBODIMENTS

The description set forth below with the appended drawings is intended as a description of presently preferred embodiments of the disclosure and is not intended to represent the only forms in which the present disclosure can be constructed and/or utilized. The description sets forth the functions and the sequence of steps for constructing and operating the disclosure. It is to be understood, however, that the same or equivalent functions and sequences can be accomplished by different embodiments that are also intended to be encompassed within the spirit and scope of this disclosure.

As discussed above, tangential flow filtration using a hollow fiber filter, such as a dialysis filter, has previously been used for water sample concentration. Dialysis filters have the advantage of being low cost, compact, and have a higher surface area (e.g. 1.5-2.5 m²) for filtration. In a typical example of tangential flow filtration, the water to be concentrated is drawn up via a pump and into the filter entrance. The water is then distributed into the individual fibers of the filter. Once in the fibers, the water will either cross the fiber wall and exit the filtrate port or else the water will go along the inside length of the fibers, out of the filter retentate exit port, and be recycled back into the system. The recycling of some of the water back into the system rather than crossing the fiber wall, is what distinguishes tangential flow filtration from dead end filtration where all water entering the filter crosses the filter wall and leaves the system as filtrate. In tangential flow filtration, a percentage of water flows tangentially, along the inside of the fibers and does not cross the fiber walls. This type of flow serves to scour the wall of the filter fibers and results in reduced fouling at the water/filter wall interface. Over time, the majority of the water will exit the filtrate port, but the fraction of the water retained (i.e., retentate) will contain the microorganisms in the original water sample.

The present invention is based on tangential flow filtration but is novel because it has been reconfigured to allow concentration of water laden with particulate matter. Despite the advantages of dialysis filters, one of their drawbacks is the inside diameter of the individual fibers is small (e.g. 0.180 to 0.200 mm), and as a consequence, during the concentration process of turbid water, particulate matter will build up in the form of a cake at the entrance to the filter's fibers at the inlet filter header.

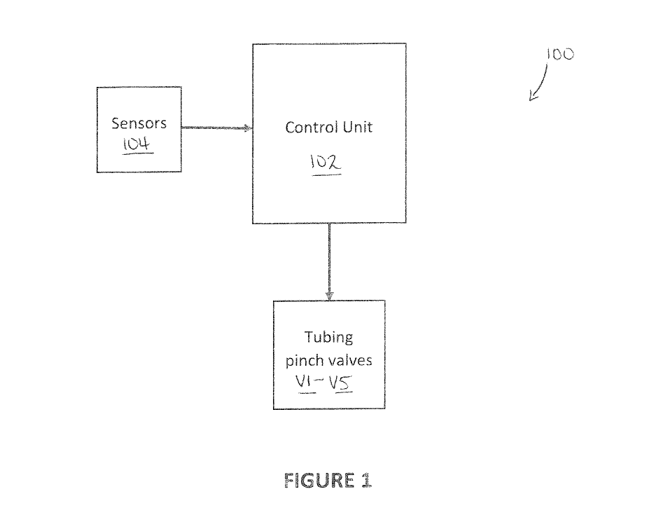

FIGS. 1-12 together show a device and method for concentrating microorganisms in large volumes of turbid water. The device may be operated manually or the device may be implemented in a computerized system for semi-automatic operation of the device 10. Referring to FIG. 1, the system 100 may comprise the device for concentrating microorganisms (hereinafter device 10) that is coupled to a computer having software therein for controlling the device 10 and a control unit 102. The device 10 may have and one or more sensors 104 (e.g. pressure transducer 20, flow meter 26, load cell 32, etc.) that may sense data regarding flow, pressure, and/or weight and may communicate the data to the control unit 102. The control unit 102 may be coupled to a plurality of electric solenoid tubing pinch valves V1-V5 in order to control their opening and closing, depending upon the data received from the sensors 104. Overall, the control unit 102 may control the valves V1-V5 and the speed and direction of the pump 16.

In the current invention, as build-up continues due to the accumulation of particulate matter at the filter inlet 23, the pressure at the filter inlet 23 will increase. If the pressure increases to a pre-determined value, the filter 22 may be backflushed with the same pump 16 used to draw up the sample. There may be a reservoir 24 which contains a small volume (˜100 ml) of filtrate. This filtrate serves as backflush fluid. During backflushing, the pump 16 is reversed, the appropriate tubing pinch valves (any of V1-V5) are opened and closed, and the backflushing fluid removes accumulated particulate matter that had built up in the inlet filter header 36. This backflush fluid is then sent to a retentate sample container 30, which is typically off-line except for backflushing and final sample recovery. For prior art that uses dialysis filters, there is no effective way to remove build-up of material at the inlet filter header 36. The present invention addresses this need.

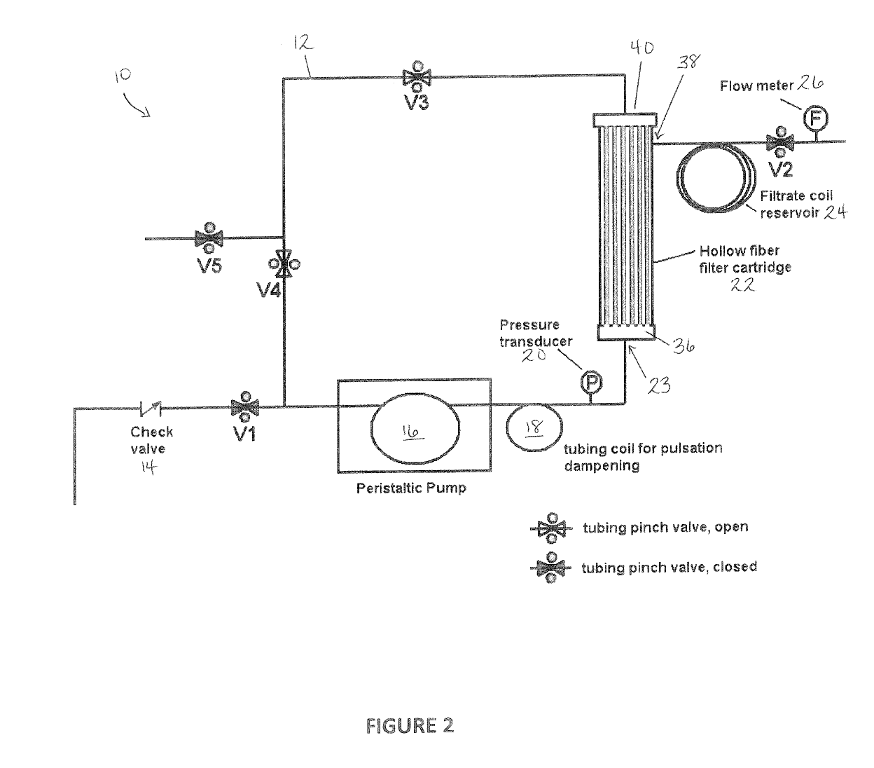

Referring to FIG. 2, an embodiment of the device 10 is shown. The device 10 comprises flexible tubing 12, a pump 16 (such as a peristaltic pump) that may be reversible and have variable speeds, and a filter 22 (such as a hollow fiber filter cartridge). The device 10 may also comprise a plurality of valves (e.g. a check valve 14 and several tubing pinch valves V1-V5) to control the flow of liquids throughout the device 10. As shown, a check valve 14 (or any other type of one-way valve) may be used to control the flow of liquids into the device 10 and to prevent the liquids from flowing out of the device 10. The tubing pinch valves V1-V5 may be electric solenoid valves that are connected to the computer having software and a control unit 102 for controlling the device 10. The device 10 may also comprise a tubing coil pulsation dampener 18 (or any other suitable pulsation dampener 18 coupled proximate a discharge side of the pump 16) and a pressure transducer 20 positioned between the peristaltic pump 16 and the hollow fiber filter cartridge 22. The device 10 may also have a filtrate coil reservoir 24 (or any other suitable filtrate reservoir 24) where a portion of the filtrate collects before it is used for backflushing. The device 10 may also have a flow meter 26. Here, tubing pinch valves V1, V2, and V5 are shown closed while tubing pinch valves V3 and V4 are shown open.

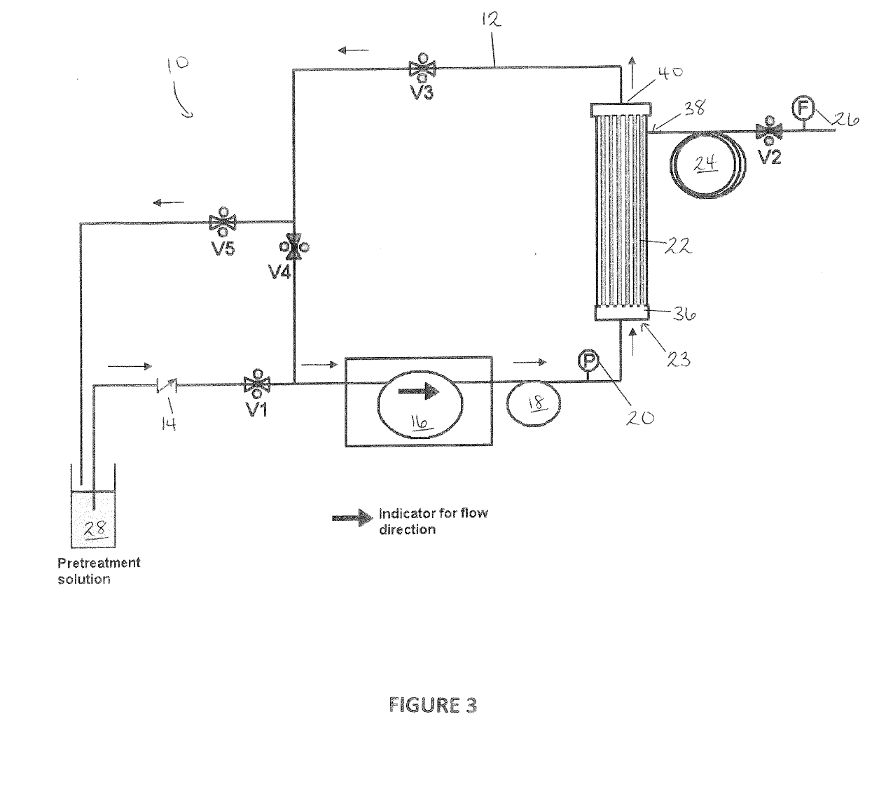

As shown in FIG. 3, the device 10 may first be primed. During this step, tubing pinch valves V2 and V4 are closed while tubing pinch valves V1, V3, and V5 are open. A pretreatment solution 28 may be drawn up via the pump 16 through the check valve 14 and circulated through the opened tubing pinch valve V1, the peristaltic pump 16, the tubing coil pulsation dampener 18, the hollow fiber filter cartridge 22, the opened tubing pinch valves V3 and V5, and out of the device 10. In this step, the pretreatment solution 28 coats the interior of the fibers of the filter 22 and the tubing 12. This step may also help to purge the device 10 of any air that may be present within the device 10. An example of pretreatment solution 28 may be 0.055% polysorbate 80 (e.g. TWEEN® 80), 0.001% antifoam (e.g. Antifoam A), and 0.1% sodium polyphosphate. It should be clearly understood, however, that substantial benefit may be obtained from the use of other suitable pretreatment solutions 28.

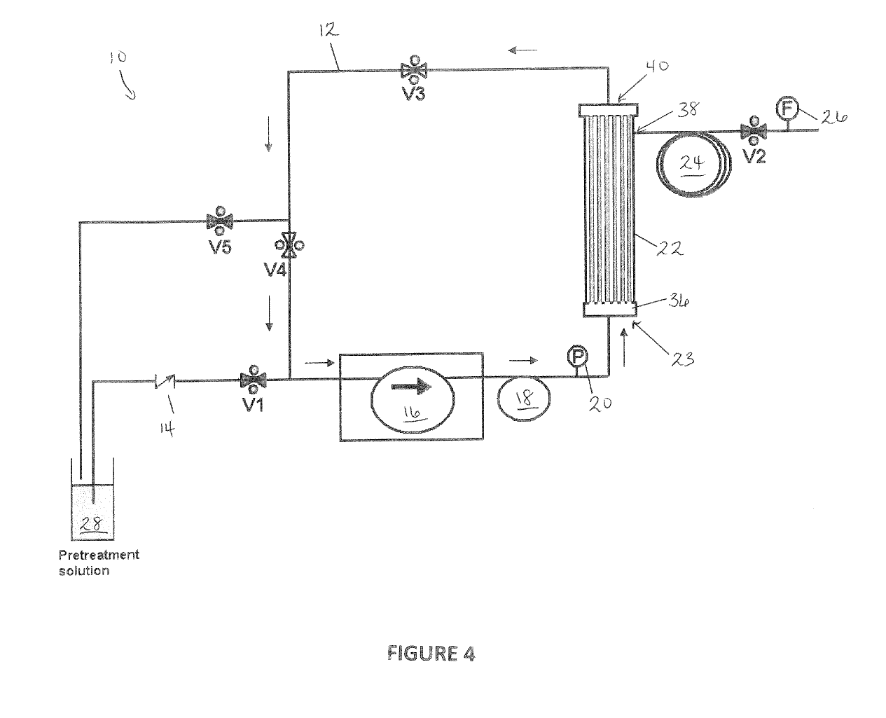

Referring to FIG. 4, after the air has been purged from the device 10, the pretreatment solution 28 may then be recirculated through the device 10. As an example, approximately 0.5-1 L of the pretreatment solution 28 may be used. But it should be clearly understood that any suitable amount of pretreatment solution 28 may be used. In this step, tubing pinch valves V1, V2, and V5 are closed while tubing pinch valves V3 and V4 are open. The pretreatment fluid 28 may be recirculated repeatedly through the peristaltic pump 16, the tubing coil pulsation dampener 18, the hollow fiber filter cartridge 22, and the opened tubing pinch valves V3 and V4 for a predetermined period of time (e.g. 3 minutes). In this step, the filter's 22 fibers and other wetted surfaces are coated with the pretreatment solution 28 in order to help lessen the sticking of microorganisms to the wetted surfaces. The polysorbate 80 (e.g. TWEEN® 80) makes the wetted surfaces hydrophilic, and since microorganisms are typically hydrophobic, the microorganisms would thus be repelled by a hydrophilic surface. The antifoam (e.g. Antifoam A) helps to reduce the foam production due to the polysorbate 80, which is a surfactant. And the sodium polyphosphate helps to make the wetted surfaces electronegative. Microorganisms are typically negatively charged and thus would be repelled by a negatively charged surface. The pretreatment solution 28 helps to maximize recovery of the microorganisms.

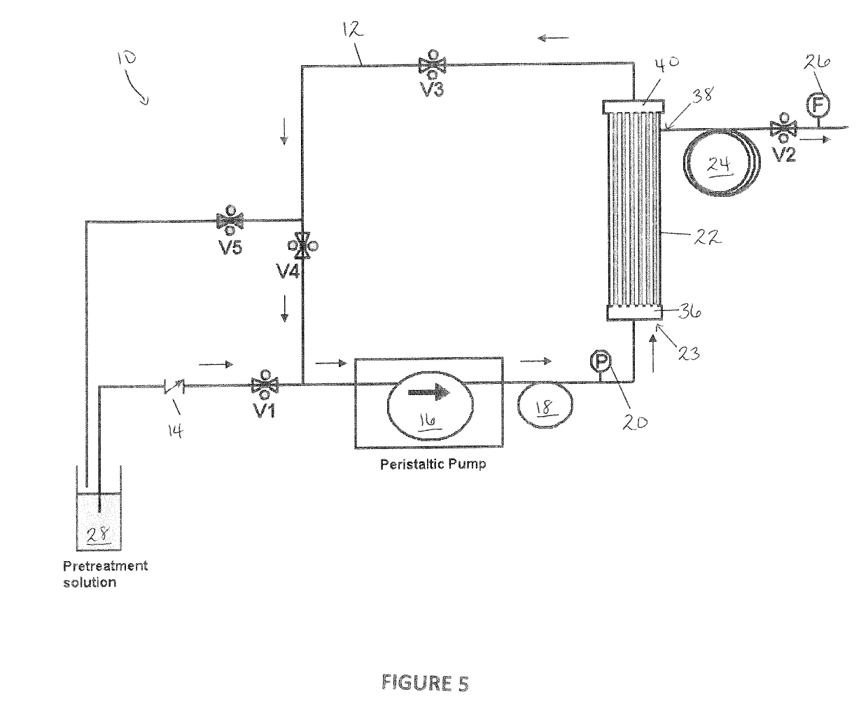

After recirculation, the pretreatment solution 28 may then be removed from the device 10. As shown in FIG. 5, tubing pinch V5 is closed while tubing pinch valves V1, V2, V3, and V4 are open. The recycled pretreatment solution 28 may pass through the peristaltic pump 16, the tubing coil pulsation dampener 18, enter the hollow fiber filter cartridge 22, cross the fiber wall, and exit the filtrate port as filtrate. Some of the pretreatment solution 28 may flow along the inside length of the fibers, out of the filter retentate exit port 40, through opened tubing pinch valves V3 and V4, and again through the peristaltic pump 16 and the hollow fiber filter cartridge 22. Eventually the pretreatment solution 28 will cross the fiber wall and exit the filtrate port 38. Alternatively, the pretreatment solution 28 may simply be removed during the normal processing of the sample 42, as described below.

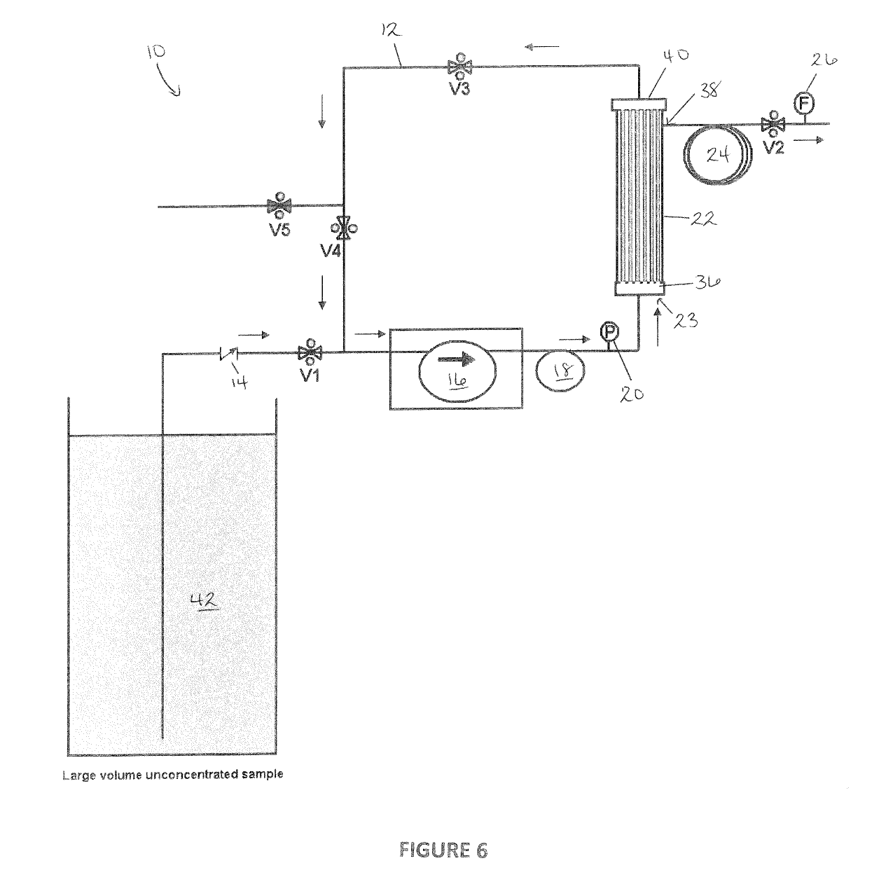

Sample concentration may then begin. In this step, shown in FIG. 6, tubing pinch valve V5 is closed while tubing pinch valves V1, V2, V3, and V4 are open. Water from the large volume unconcentrated sample 42 may be drawn up via the pump 16 through the check valve 14 and circulated through the opened tubing pinch valve V1, the peristaltic pump 16, the tubing coil pulsation dampener 18, and into the filter inlet 23 of the hollow fiber filter cartridge 22. The water is then distributed into the individual fibers of the hollow fiber filter cartridge 22. Once the water is in the fibers, the water may either cross the fiber wall and exit as filtered water through the filtrate port 38 or, alternatively, the water may flow along the inside length of the fibers, out of the filter retentate exit port 40, through opened tubing pinch valves V3 and V4, and be recycled back through the device 10. Over time, the majority of the water will exit the filtrate port 38. This process may continue until enough of the water sample has been concentrated or until enough particulate matter has accumulated so that backflushing is required.

The area of the device where particulate matter may typically accumulate is at the inlet filter header 36. With other technologies, efforts have been made to keep particulate matter from entering this inlet filter header 36. However, with the present invention, the step of backflushing particulate matter off of the inlet filter header 36 allows the inlet filter header 36 itself to function as a pre-filter for individual filter fibers by preventing particulate matter larger than the fiber diameter from entering the fibers. And while particulate matter accumulates to block the entrances, it can also be removed by backflushing. Typically, the fiber diameter for single-use hollow fiber filters is about 0.180-0.200 mm. Other types of hollow fiber filters may be available that have fiber diameters that are large enough so that the fiber entrances will not be as likely to clog; however, those other filters may have smaller filtration surface area and may be cost prohibitive for single-use applications.

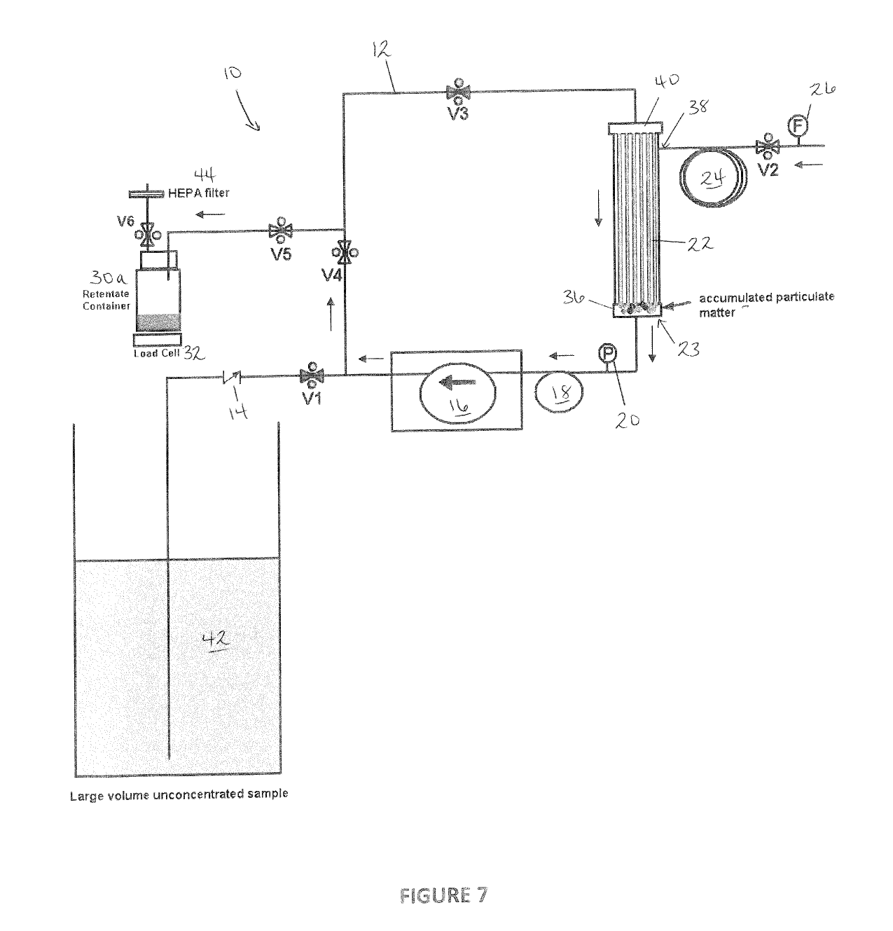

During the backflushing process, as shown in FIG. 7, tubing pinch valves V1 and V3 are closed while tubing pinch valves V2, V4, and V5 are open. The peristaltic pump 16 in this step pumps in the opposite direction so that fluid that has accumulated in the filtrate coil reservoir 24 is drawn back through the fiber walls, through the interior of the fibers, and into the inlet filter header 36 where accumulated particulate material is dislodged. The spent backflushing fluid, which now carries the dislodged particulate matter, is then directed through open tubing pinch valves V4 and V5 to the retentate sample container 30a (referred to generically as retentate sample container 30). When enough fluid from the filtrate coil reservoir 24 has been drawn up (typically 100 mL), the peristaltic pump 16 may be turned off and the fluid communication between the concentration device 10 and the retentate sample container 30a may be disconnected by closing tubing pinch valve V5. A load cell 32 may be used to measure the amount of retentate collected in the retentate sample container 30a. In order to minimize bioaerosol generation, the retentate sample container 30a may be closed to the atmosphere. For filling, the retentate sample container 30a may have a vent that is open via a pinch valve V6. Vented air may pass through a High Efficiency Particulate Air (HEPA) filter 44 prior to release, which typically accompanies the two-port retentate sample container 30a.

After backflushing, the sample concentration step as described above and shown in FIG. 6, may resume and continue. The sequence of sample concentration and backflushing may be repeated until the retentate sample container 30a is filled to a predetermined percentage of maximum container capacity, or until the volume of retentate is determined to be as large as practical.

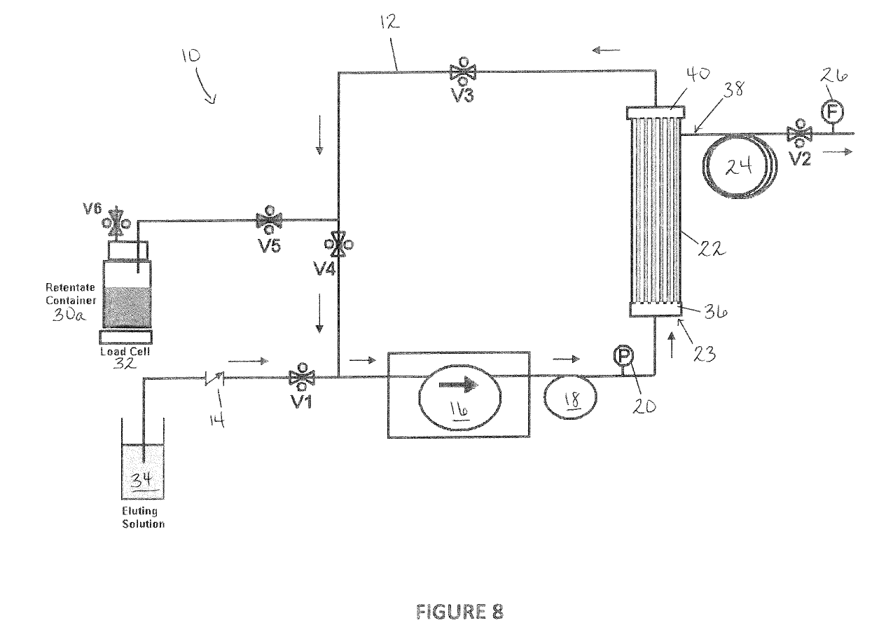

When enough of the original sample has been concentrated, elution may then commence. As shown in FIG. 8, tubing pinch valve V5 is closed while tubing pinch valves V1, V2, V3, and V4 are open. The pinch valve V6 of the retentate sample container 30a may also be closed. Eluting solution 34 is drawn up and processed the same way that the unconcentrated sample 42 was processed. After a predetermined volume of the eluting solution 34 has been drawn up, the process may stop. The eluting solution 34 may then be recirculated in a fashion similar to the pretreatment solution 28 as that shown in FIG. 4. An example of the eluting solution 34 may be 0.001% polysorbate 80 (e.g. TWEEN® 80). The eluting solution 34 helps to loosen microorganisms that may have adhered to the wetted surfaces during the concentration process and may result in significantly more microorganisms being captured in the final concentrated sample/retentate.

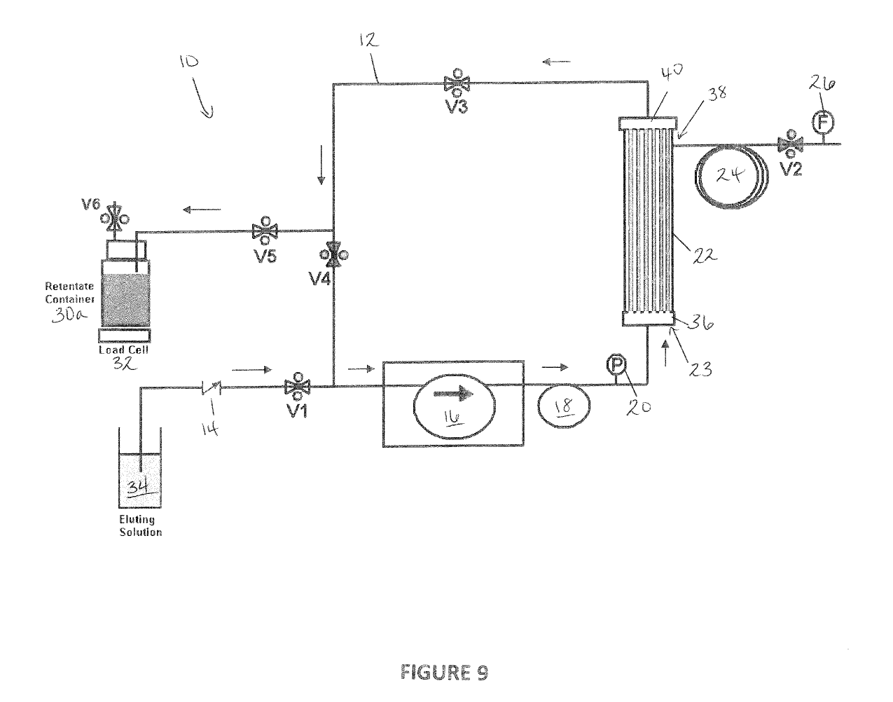

After recirculation, the retentate sample may be recovered. FIG. 9 shows a forward flush wherein the eluting solution 34 is used to flush the hollow fiber filter cartridge 22 in a forward direction so that the retentate water in the hollow fiber filter cartridge 22 and tubing 12 is sent to the retentate container 30a. Here, the tubing pinch valves V2 and V4 are shown closed while tubing pinch valves V1, V3, and V5 are open. Pinch valve V6 of the retentate container 30a may also be open. Alternatively, the retentate sample may be recovered via backflushing. In the event that backflushing is used, the process is similar to that shown in FIG. 7 except, by this time in the process, the fluid in the filtrate coil reservoir 24 is filtered eluting solution 34.

Other technologies typically require a more complex three-port retentate sample container. Or, other technologies require that the unconcentrated sample container also serve as the retentate sample container. However, the present invention may use a retentate sample container 30 that requires only one port (e.g. retentate sample container 30b) or two ports (e.g. retentate sample container 30a).

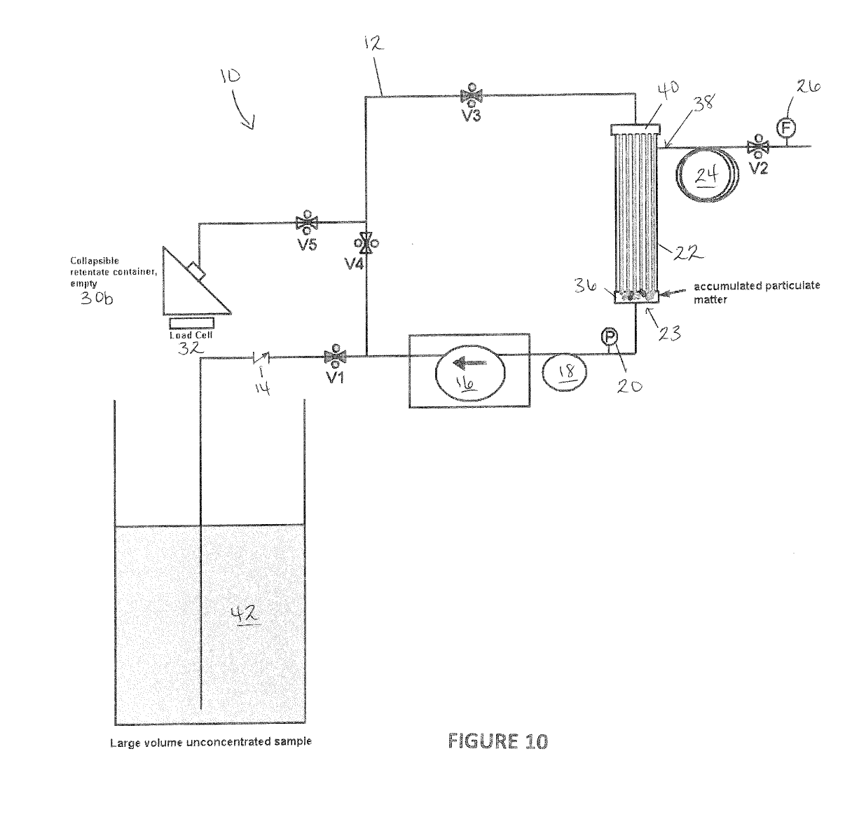

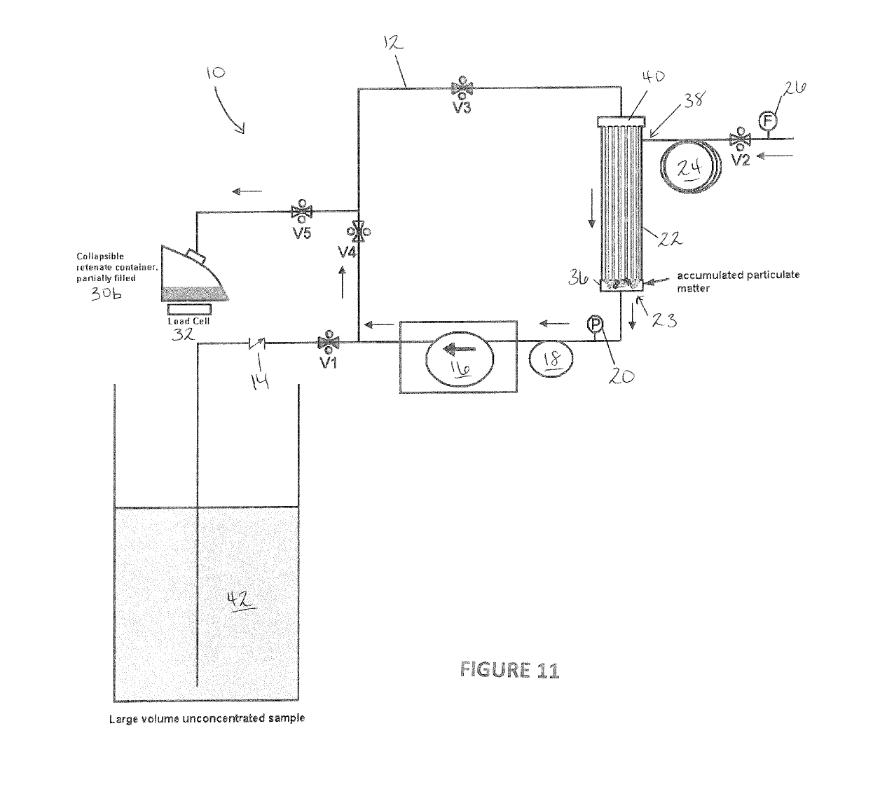

In the embodiments shown in FIGS. 7-9, the retentate sample container 30a is a bottle with two ports: one for retentate sample delivery and the other for venting during filling. In alternative embodiment, as shown in FIGS. 10-11, the retentate sample container 30b (referred to generically as retentate sample container 30) may be collapsible with a single port. At the beginning of retentate sample collection, the collapsible retentate sample container 30b would be fully collapsed and will expand as retentate sample is added. In this embodiment, there would be no need for the collapsible retentate sample container 30b to be vented. FIG. 10 shows an empty collapsible retentate sample container 30b coupled to the device 10 just before backflushing with the tubing pinch valves V1 and V3 closed while tubing pinch valves V2, V4, and V5 are open. FIG. 11 shows a partially filled collapsible retentate sample container 30b coupled to the device 10 during backflushing with the tubing pinch valves V1 and V3 closed while tubing pinch valves V2, V4, and V5 are open. An advantage to this embodiment is that foamy retentate samples do not cause operational problems. In embodiments where more than one port is used in the retentate container, foam could escape through the vent port.

The method for concentrating microorganisms in large volumes of turbid water may occur under ambient temperature and pressure. It may be preferred that the maximum temperature should be less than 40° C. so that target organisms may not be adversely affected. It may also be recommended that the minimum temperature be greater than 0° C. in order to prevent the water from freezing. It may also be preferred that the pressure at the pump outlet (the point of highest pressure) be controlled so that it is less than 30 psig.

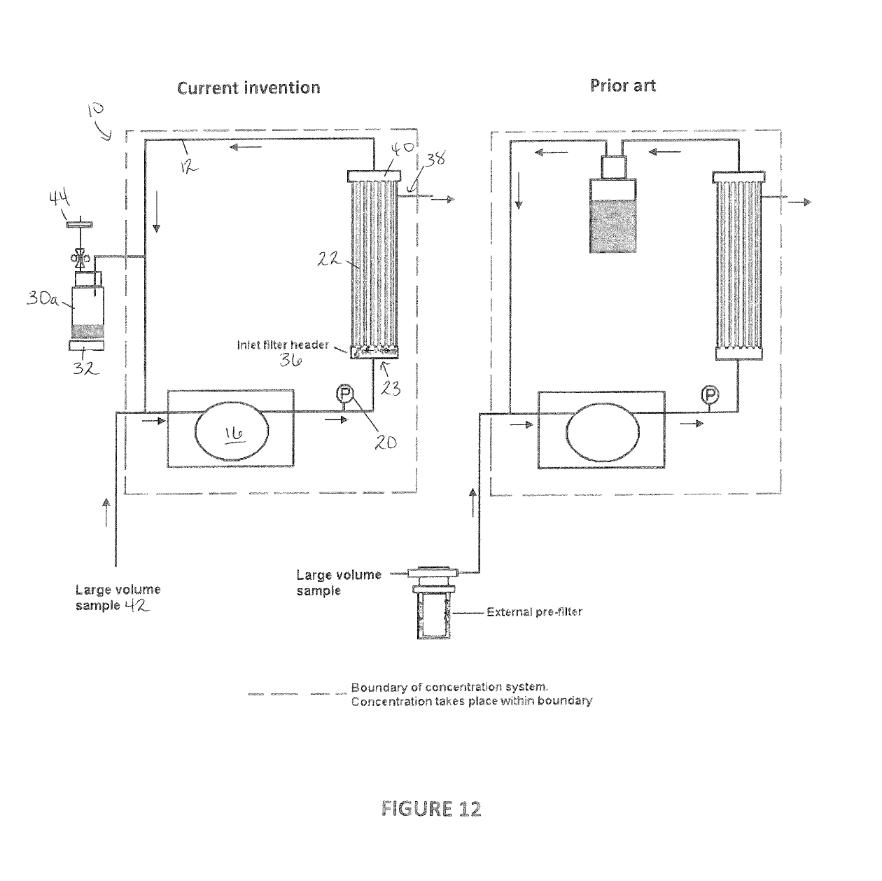

FIG. 12 shows a comparison between the present invention and other technologies where pre-filtration occurs. As mentioned above, other technologies like the WSC have added pre-filters to their sample inlets in an attempt to minimize clogging. However, in those technologies, the pre-filter is outside of the concentration device 10; thus, there is the potential of losing target organisms in the pre-filter. Furthermore, if the pre-filter accumulates enough particulate material, there is no efficient way to clean off the clogged surface. Still further, placing a pre-filter on the vacuum side of the pump in those technologies would create a more complex procedure for field operations; e.g. a greater volume of pretreatment solution would be required due to the necessity of coating the internal surfaces of the pre-filter. There would also be a greater possibility for vacuum leaks. Finally, the pump typically used in those technologies is a peristaltic pump which yields substantially worse performance with increasing suction head which would be encountered when that type of pre-filter becomes coated with particulate matter. A peristaltic pump may be used in the present invention, but there is no pre-filter at the suction side of the pump 16; thus, in the present invention, a peristaltic pump has proven to be effective.

In the present invention, the inlet filter header 36, where clogging may occur, effectively acts as a pre-filter. But, in the present invention, the pre-filter is a part of the concentration device 10 rather than outside as described above in the other technologies. Furthermore, in the present invention, the pre-filter is on the pressure side of the peristaltic pump 16, which is advantageous with regard to pump efficiency. For the hollow fiber filter cartridge 22 used in the present invention and with other technologies, the filtration occurs at the microscopic pores along the walls of the fibers and the entrances to the fibers at the inlet filter header 36 typically only serve as the conduit for the water to reach the fibers' interior. However, with turbid water samples, particulate matter larger than the fiber diameter gets caught at the entrances to the individual fibers. And although this prevents large particles from entering the actual fibers, the particulate matter builds up as cake in the filter header, which causes operational problems such as pressure build-up and decreased flow.

The present invention allows filtrate water to be backflushed through the hollow fiber filter cartridge 22 to dislodge this accumulated material which is then jettisoned from the main concentration process and stored off-line in the retentate sample container 30. Once dislodged and jettisoned, the particulate matter does not get reintroduced into the hollow fiber filter cartridge 22, but remains isolated in the retentate sample container 30. With the other technologies, there is no effective way to backflush the inlet filter header 36 during the concentration process. This is because the retentate containers 30 of the other technologies are positioned in-line with the main concentration process; thus, backflushed particulate matter that has been removed will eventually be drawn back into the device and will cause the same clogging problems experienced before.

The foregoing description is provided to enable any person skilled in the relevant art to practice the various embodiments described herein. Various modifications to these embodiments will be readily apparent to those skilled in the relevant art, and generic principles defined herein can be applied to other embodiments. Thus, the claims are not intended to be limited to the embodiments shown and described herein, but are to be accorded the full scope consistent with the language of the claims, wherein reference to an element in the singular is not intended to mean “one and only one” unless specifically stated, but rather “one or more.” All structural and functional equivalents to the elements of the various embodiments described throughout this disclosure that are known or later come to be known to those of ordinary skill in the relevant art are expressly incorporated herein by reference and intended to be encompassed by the claims. Moreover, nothing disclosed herein is intended to be dedicated to the public.

What is claimed is:

1. A device for concentrating microorganisms in a water sample:
a line of tubing;
a reversible pump;
a filter;
an inlet filter header coupled to an inlet of the filter;
a plurality of valves for controlling the flow of liquids through the tubing; and
a filtrate reservoir for collecting filtrate after the filtrate has exited a filtrate port of the filter,
wherein the inlet filter header is adapted to be used as an in-line pre-filter for the device.

2. The device of claim 1 further comprising:
at least one sensor configured to capture at least one of flow rate data, water pressure data, and weight data; and
a control unit coupled to the sensor for processing the data captured by the sensor in order to control at least one of the plurality of valves and the pump.

3. The device of claim 1 further comprising an off-line retentate sample container for collecting retentate after the filter has been backflushed with the filtrate from the filtrate reservoir that flows back from the filtrate reservoir through the filter and out through the inlet filter header.

4. The device of claim 4 wherein the retentate sample container is collapsible.

5. The device of claim 1 wherein the pump is a peristaltic pump.

6. The device of claim 5 wherein the pump further comprises a pulsation dampener coupled proximate a discharge side of the peristaltic pump.

7. The device of claim 1 wherein the filter is a hollow fiber filter cartridge.

8. A method for concentrating microorganisms in a water sample comprising the steps of:
providing a device for concentrating microorganism in a water sample;
pumping the water sample through the device in a forward direction;
collecting particulate matter from the water sample within an in-line pre-filter;
backflushing the pre-filter with filtrate pumped through the device in a reverse direction;
collecting retentate in an off-line retentate sample container, wherein the retentate contains the particulate matter; and
isolating the particulate matter within the off-line retentate sample container in order to prevent the particulate matter from being recirculated through the device.

9. The method of claim 8 wherein the device comprises:
a line of tubing;
a variable speed, reversible pump for controlling the flow of liquids through the tubing;
a filter;
an in-line pre-filter coupled to an inlet of the filter, wherein the in-line pre-filter is an inlet filter header;
a plurality of valves for controlling the flow of liquids through the tubing;
a filtrate reservoir for collecting filtrate after the filtrate has exited a filtrate port of the filter; and
a retentate sample container coupled off-line to the device for collecting retentate.

10. The device of claim 9 wherein the device further comprises:
a pressure transducer to sensing pressure within the device;
a flow meter for sensing flow fate within the device;
a load cell for sensing weight of the retentate within the retentate sample container; and
a control unit coupled to the pressure transducer, the flow meter, and the load cell, wherein the control unit processes data captured by at least one of the pressure transducer, the flow meter, and the load cell in order to control at least one of the plurality of valves and the pump.

11. The method of claim 8 further comprising the step of priming the device with pretreatment solution in order to remove air from the device before pumping the water sample through the device.

12. The method of claim 11 further comprising the step of recirculating the pretreatment solution through the device in order to coat interior surfaces of the device with a hydrophilic solution.

13. The method of claim 8 further comprising the step of eluting the retentate by pumping eluting solution through the device in a forward direction.

14. The method of claim 13 further comprising the step of recirculating the eluting solution through the device in order to loosen microorganisms that have stuck to the interior surfaces of the device.

15. The method of claim 14 wherein the eluting solution is hydrophilic.

16. The method of claim 9 wherein the filter is a hollow fiber filter cartridge.

17. The method of claim 9 wherein the pump is a peristaltic pump.

18. A method for concentrating microorganisms in a water sample comprising the steps of:
providing a device for concentrating microorganisms in a water sample, the device comprising:
a line of tubing;
a variable speed, reversible peristaltic pump;
a hollow fiber filter cartridge;
an inlet filter header coupled to an inlet of the filter;
a plurality of valves for controlling the flow of liquids through the tubing;
a filtrate reservoir; and
a retentate sample container coupled off-line to the device for collecting retentate;
priming the device with pretreatment solution in order to remove air from the device;
recirculating the pretreatment solution through the device in order to coat filter fibers and tubing with a hydrophilic solution that will prevent the microorganisms from sticking to the filter fibers and to the tubing;
pumping the water sample through the device in a forward direction;
collecting an amount of filtrate within the filtrate reservoir after the water has exited a filtrate port of the hollow fiber filter cartridge;
backflushing the filtrate in a reverse direction from the filtrate reservoir back through the hollow fiber filter cartridge and out the inlet filter header in order to dislodge particulate matter that has collected at the inlet filter header;
collecting retentate within the retentate sample container; and
pumping hydrophilic eluting solution through the device.

19. The method of claim 18 further comprising the step of isolating the retentate within the retentate sample container in order to prevent particulate matter within the retentate from being reintroduced into the hollow fiber filter cartridge.

20. The method of claim 18 further comprising the steps of:
recirculating the eluting solution through the device in order to loosen microorganisms that have stuck to the filter fibers and to the tubing; and
forward flushing the hollow fiber filter cartridge with eluting solution in a forward direction so that the retentate is sent to the retentate sample container.
